(19) United States
(12) Patent Application Publication
(10) Pub. No.: US 2025/0009211 A1
(43) Pub. Date: Jan. 9, 2025

(54) Optical Instrument with Articulating Image Sensing Apparatus and Working Channel

(71) Applicant: KARL STORZ Endovision, Inc., Charlton, MA (US)

(72) Inventor: Vernon Hopkins, Worcester, MA (US)

(73) Assignee: KARL STORZ Endovision, Inc., Charlton, MA (US)

(21) Appl. No.: 18/892,074

(22) Filed: Sep. 20, 2024

Related U.S. Application Data
(63) Continuation of application No. PCT/US2022/021108, filed on Mar. 21, 2022.

(51) Int. Cl. A61B 1/008; A61B 1/00; A61B 1/05; A61B 1/06

(52) U.S. Cl. CPC A61B 1/008; A61B 1/00149; A61B 1/05; A61B 1/0676

(57) ABSTRACT

An imaging apparatus is configured for use with an instrument having an instrument shaft and transparent distal end portion with a working channel opening to a working channel tube. The imaging apparatus includes an image sensor assembly, a first articulating structure, and a second articulating structure mounted on the first articulating structure. The first articulating structure is positioned within the instrument shaft in an operating position for rotation about a longitudinal axis (L) of the distal end portion. The image sensor assembly has an imaging axis (I) and is mounted on the second articulating structure to facilitate rotation of the imaging axis (I) about a lateral articulation axis (T1, T2) extending transverse to the longitudinal axis (L) of the distal end portion. Throughout this rotation the image sensor assembly remains within a sensor area defined between the working channel tube and in internal surface of the distal end portion.

### CROSS REFERENCE TO RELATED APPLICATIONS

This application is a continuation of PCT Application No. PCT/US2022/021108 filed Mar. 21, 2022, and entitled “Optical Instrument with Articulating Image Sensing Apparatus and Working Channel,” which is incorporated herein by reference.

### TECHNICAL FIELD

Exemplary features relate to optical instruments such as endoscopes and borescopes having an image sensor assembly at the distal end of the instrument shaft. More particularly, some exemplary features of the disclosure relate to optical instruments that can produce an image from a wide range of orientations within the distal end of the instrument shaft and that also provide a working channel through the instrument.

### BACKGROUND

Instruments such as endoscopes and borescopes are used to allow a visual inspection of locations that are not readily accessible. For example, endoscopes are used in medical applications to provide a view of an area within a patient's body. Whether employed for medical or other applications, the instrument typically includes an elongated shaft of relatively small diameter extending from a handle to a distal end. An imaging or viewing arrangement is included with the instrument to allow a user to obtain a view from the shaft distal end. This arrangement may include a system of lenses and a light conduit through the shaft to direct an image from the distal end to an eyepiece associated with the instrument handle. Alternatively, the imaging or viewing arrangement may include an electronic imaging device at the distal end of the instrument shaft. Such an electronic imaging device collects image data and communicates that data through the shaft and handle ultimately to a processing system that assembles the data to produce an image displayed on a suitable display device.

Depending upon the procedure for which the instrument is used, it may be necessary for the operator to view a relatively large area or view a relatively small area from different angles. In a medical procedure for example, the operator may desire to view a location that is larger than the field of view of the imaging collecting arrangement of the endoscope or view a location from different angles. In these situations it has been necessary for the endoscope operator to move the distal end of the endoscope in an effort to provide the desired views, and sometimes move the distal end repeatedly in given area.

Endoscopes have been developed to give the operator the ability to adjust viewing angle. U.S. Patent Application Publication No. 2015/0238068 discloses an endoscope having an objective lens and prism that is mounted on a pivotable structure at the distal end of the endoscope. This endoscope, however, allows rotation to only one side of the device. Thus the endoscope had to be repositioned in the area of the procedure in order to view a location on the opposite side of the endoscope shaft. U.S. Patent Application Publication No. 2014/0012080 shows another endoscope with an image collecting part that may be tilted to one side of the endoscope at the distal end. This arrangement also requires the endoscope distal end to be repositioned to obtain views of areas on the opposite side of the endoscope shaft (that is, opposite the side to which the image collecting device is tilted at a given point in time).

U.S. Pat. No. 6,371,909 discloses an endoscope having an imaging assembly mounted in the distal end of the endoscope so as to allow articulation about two axes. This two-axis articulation facilitates different viewing angles through a transparent cover at the distal end of the endoscope. The articulating arrangement disclosed in U.S. Pat. No. 6,371,909, however, greatly constrained the nature and size of the imaging device which could be employed for a given diameter endoscope distal end. Also, the distal end of the endoscope in U.S. Pat. No. 6,371,909 had to be repositioned to provide a view of any area other than the area at the far distal tip of the endoscope shaft.

U.S. Pat. No. 10,517,470 discloses an optical instrument having a transparent end section at the distal end of the endoscope. An imaging assembly is mounted within the transparent end section and is capable of articulation about two axes. While the optical instrument disclosed in U.S. Pat. No. 10,517,470 allows substantially the entire area around the transparent distal end section to be imaged without having to change the position of the distal end section, the optical instrument did not allow any procedure to be performed through the endoscope. Thus in order to perform any procedure in the area being imaged it was necessary to either insert another endoscope into the area or otherwise provide access to the site.

There remains a need in the art to provide an optical instrument such as an endoscope or borescope that allows the imaging device to be adjusted so that different views can be obtained without having to move the instrument distal end and while providing access to the area of the imaged site to perform various procedures.

### SUMMARY

According to one exemplary configuration, an optical instrument is disclosed, such as an endoscope or borescope, having an image sensor that can be articulated within the instrument shaft about both a longitudinal axis and a lateral articulation axis while also providing a working channel to the distal end of the instrument.

An imaging apparatus according to one aspect of the disclosed technology is adapted for use in an optical instrument such as an endoscope or borescope having an elongated shaft with a transparent distal end portion. Such an optical instrument will be referred to in this disclosure and the accompanying claims simply as an “instrument,” and this term is intended to encompass endoscopes, borescopes, and similar optical instruments.

An imaging apparatus according to this first aspect of the disclosed technology is configured for use with an instrument having an elongated instrument shaft having a distal end portion that is transparent and includes a working channel opening to a working channel tube included within the instrument shaft. Such an imaging apparatus includes an image sensor assembly, a first articulating structure, and a second articulating structure mounted on the first articulating structure. The first articulating structure is adapted to be positioned within the instrument shaft in an operating position for rotation about a longitudinal axis of the distal end portion of the instrument shaft. The image sensor assembly has an imaging axis and is mounted on the second articulating structure to facilitate rotation of the imaging axis about a lateral articulation axis extending transverse to the longitudinal axis of the distal end portion of the instrument shaft. Throughout this rotation of the imaging axis about the lateral articulation axis while the first articulating structure is in the operating position within the instrument shaft, the image sensor assembly remains within a sensor area defined between an external surface of the working channel tube and in internal surface of the distal end portion.

The combination of the image sensor assembly mounted on the second articulating structure, which is in turn mounted on the first articulating structure, facilitates the articulation of the image sensor assembly within the instrument shaft about two axes. The articulation about these two axes allows the image sensor assembly to be positioned within the instrument so that its imaging axis is directed in any direction a full 360° about the shaft distal end portion longitudinal axis and also laterally about the lateral articulation axis preferably at least 90°, and even further in some embodiments as will be described below. Thus when the imaging apparatus is mounted in an instrument, the instrument is capable of providing an overall field of view over a relatively large area without having to move the distal end portion of the instrument and without interfering with a working channel of the instrument through which various procedures may be performed in the area of the site being imaged.

When the imaging apparatus is mounted in the operating position within an instrument shaft, an articulation control assembly may be used to control the articulation of the image sensor assembly. In particular, the articulation control assembly may be used to control the rotation of the first articulating structure, and thus the image sensor assembly, about the distal end portion longitudinal axis. The articulation control assembly may also be used to control the articulation of the image sensor assembly, and thus the imaging axis, about the lateral articulation axis. As will be described below in connection with the representative embodiments, any suitable arrangement may be employed in the articulation control assembly to effect the desired movement of the image sensor assembly, including various types of motors and mechanical linkages to the image sensor assembly.

The first articulating structure in some implementations of an imaging apparatus may comprise an axial articulation tube adapted to be positioned within the instrument shaft with the longitudinal axis of the tube aligning with the longitudinal axis of the distal end portion of the instrument shaft. In these implementations, the axial articulation tube mounted within the instrument shaft may terminate short of the distal end of the instrument shaft provided the image sensor assembly is positioned appropriately within the transparent end portion of the shaft. Otherwise, the axial articulation tube may include a distal end section that is adapted to extend distally past the image sensor assembly in the instrument shaft when the axial articulation tube is mounted in the operating position. In these embodiments the distal end section of the elongated tube includes a transparent part over at least a field of view range for the image sensor assembly about the lateral articulation axis. In either case, an axial articulation tube comprising the first articulating structure may include a proximal end adapted to extend to a handle of the instrument when the imaging apparatus is mounted in the operating position. The proximal end of the tube may be connected to a suitable rotation control device associated with the instrument handle to facilitate control of the articulation about the distal end portion longitudinal axis.

In some implementations of the articulating imaging apparatus, one or more sensor assembly light sources are mounted on the image sensor assembly. This placement of light sources for the image sensor assembly ensures proper illumination is available for imaging regardless of the orientation of the image sensor assembly within the instrument shaft. Additionally or alternatively to one or more sensor assembly light sources, an imaging apparatus according to the first aspect may include one or both of a distal tip light source and a base light source. The distal tip light source may be mounted on the first articulating structure at a distal end of the first articulating structure and a base light source may be mounted within the first articulating structure between a proximal end of the first articulating structure and lateral articulation axis.

In addition to facilitating articulation about the distal end portion longitudinal axis and the lateral articulation axis, some implementations of the imaging apparatus allow the image sensor assembly to be moved longitudinally within the shaft distal end portion. This longitudinal movement increases overall field of view for the image sensor assembly about the instrument distal end portion.

In implementations of the second articulating structure (that facilitates articulation about the lateral articulation axis) comprising the axial articulation tube, the axial articulation tube may be adapted to be mounted on the working channel tube or mounted on the instrument shaft. Where the axial articulation tube is adapted to be mounted on the working channel tube an axial articulation tube bearing structure may be supported on the working channel tube to facilitate the desired axial articulation. Where the axial articulation tube is adapted to be mounted on the instrument shaft an axial articulation tube bearing structure may be supported on the instrument shaft.

Whether the axial articulation tube is adapted to be supported on the working channel or the instrument shaft the second articulation structure may comprise a pivot base mounted on the axial articulation tube, the pivot base including a first base pivot structure and a second base pivot structure. In these implementations the first base pivot structure may cooperate with a first sensor pivot structure of the sensor assembly and the second base pivot structure may cooperate with a second sensor pivot structure of the sensor assembly to facilitate the rotation of the imaging axis about the lateral articulation axis.

Particularly in implementations where the axial articulation tube is adapted to be supported on the instrument shaft, the pivot base may be mounted on the axial articulation tube on two circumferentially spaced apart extensions that extend from a proximal portion of the axial articulation tube.

Whether the axial articulation tube is adapted to be supported on the working channel or the instrument shaft the second articulation structure comprising a pivot base may be implemented as a track-based structure. In these track-based embodiments, the first and second sensor pivot structure each includes fore and aft projections positioned at a respective lateral side of the image sensor assembly. The fore and aft projections are aligned, respectively, along fore and aft axes in these embodiments and project past a plane aligned with and extending perpendicular to the respective lateral side to be received in a respective track of the pivot base. The tracks represent the first and second base pivot structures and each includes a transverse section and a longitudinal section connected together by a curved section. The transverse sections each extend transverse to the longitudinal axis of the distal end portion of the instrument shaft when the imaging structure is in the operating position while the longitudinal sections each extend parallel to the longitudinal axis of the instrument distal end portion. This track-based arrangement allows the image sensor assembly to move to different positions along the track, which, due to the transverse and longitudinal sections, places the image sensor assembly at different angular orientations about the lateral articulation axis and effectively allowing the rotation of the imaging axis about the lateral articulation axis.

In embodiments including a track-based structure for facilitating articulation of the image sensor assembly and imaging axis about the lateral articulation axis, each track may include an inclined section at a bottom end of the respective longitudinal section. The inclined sections are each inclined so that they define a deviation greater than 90° with respect to the transverse sections. Thus the inclined sections of the tracks allow the image sensor assembly to be positioned with the imaging axis extending somewhat backwards along the instrument shaft, away from the shaft distal end.

A second aspect includes instruments including an instrument shaft, working channel tube, first articulating structure, second articulating structure, image sensor assembly, and articulation control assembly as described above in connection with the first aspect. Instruments according to this second aspect may include any of the implementation variations described above in connection with imaging apparatuses according to the first aspect.

These and other advantages and features will be apparent from the following description of representative embodiments considered along with the accompanying drawings.

### BRIEF DESCRIPTION OF THE DRAWINGS

FIG. 1 is a view in perspective of an instrument according to one aspect of the disclosed technology.

FIG. 2 is a view in perspective of the distal end of the instrument shaft shown in FIG. 1.

FIG. 3 view in section taken along line 3-3 in FIG. 2.

FIG. 4 is an end view of the image sensor assembly and a portion of the articulation track shown in FIG. 3.

FIG. 5 is a view in perspective similar to FIG. 2 but showing the image sensor assembly rotated 90° about its lateral articulation axis.

FIG. 6 is a view in section taken along line 6-6 in FIG. 5, and also showing an additional position of the image sensor assembly in phantom lines.

FIG. 7 is a view in section similar to FIG. 3 of an additional embodiment of the disclosed technology.

FIG. 8 is an end view of the image sensor assembly and pivot structure shown in FIG. 7.

FIG. 9 is a view in section similar to FIG. 7 but showing the image sensor assembly rotated 90° about its lateral articulation axis.

FIG. 10 is a view in section similar to FIG. 7 of an additional embodiment of the disclosed technology in which the axial articulation tube is supported on the working channel tube.

### DESCRIPTION OF REPRESENTATIVE EMBODIMENTS

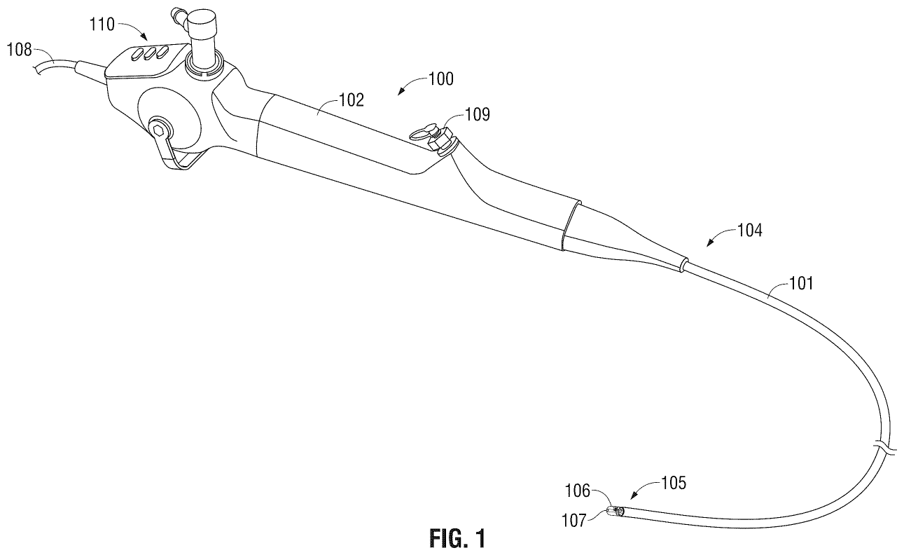

Referring to FIG. 1, an instrument 100 employing an imaging apparatus according to one aspect includes an elongated shaft 101 and a handle 102. Shaft 101 extends from a proximal end shown generally at reference numeral 104 connected to handle 102 to a distal end generally indicated at reference numeral 105. A distal end portion 106 with a working channel opening 107 is included at the shaft distal end 105. The imaging apparatus according to one aspect is located at least partially in distal end portion 106, although the assembly and the structure on which it is mounted are not shown in FIG. 1 due to the scale of the figure.

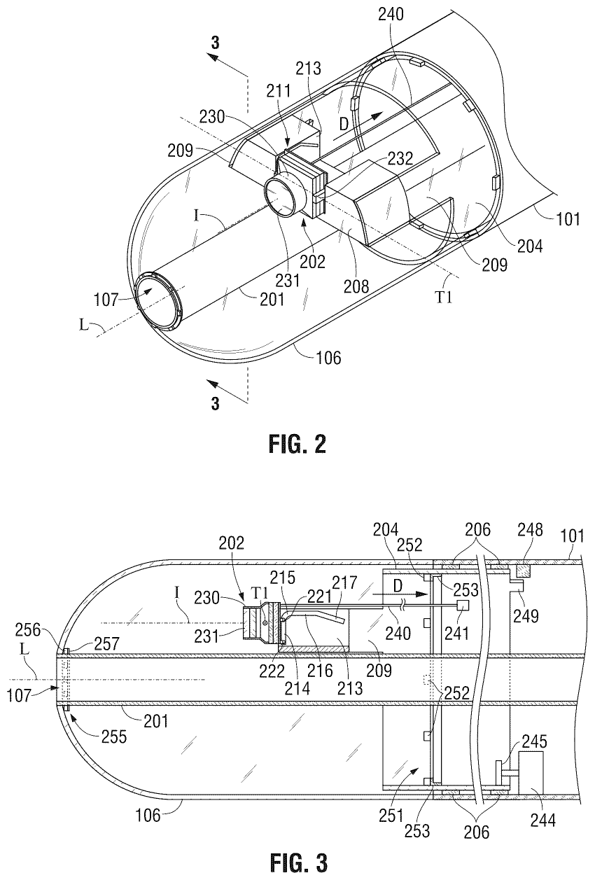

Instrument 100 receives electrical operating power through a cable 108 that extends from a proximal end of handle 102 in this example instrument. This electrical operating power may be used to operate one or more light sources and other electronic elements mounted within distal end portion 106, such as an imaging device included in the imaging apparatus. Also, data signals from such an imaging device may be communicated through appropriate conduits within shaft 101 and handle 102 to cable 108. These data signals may be communicated through cable 108 to processing equipment (not shown) that processes the image data and drives one or more video monitors to display the images collected at distal end 105 of instrument 100. Port 109 is provided in handle 102 to provide access to a working channel (201 in FIGS. 2-6) extending the length of shaft 101 to the working channel opening 107. Those familiar with endoscopes and borescopes will appreciate that instrument 100 includes a number of additional features such as controls 110 for controlling the operation of the instrument. Although controls relating to the articulating image sensor assembly will be described further below, the general operation and control of instrument 100 will not be described further herein in order to avoid obscuring the presently disclosed technology in unnecessary detail.

Referring to FIG. 2, it is apparent that distal end portion 106 of shaft 101 is transparent around its entire circumference. Working channel 201 in this particular embodiment comprises a tube that extends the entire length of instrument shaft 101 to working channel port 109 shown in FIG. 1. Working channel 201 terminates at its distal end at the working channel opening 107 formed in distal end portion 106. The periphery of working channel 201 is connected by suitable means to the working channel opening 107 to form a fluid-tight seal. A longitudinal axis of shaft distal end portion 106 is shown in FIGS. 2-6 at axis line L.

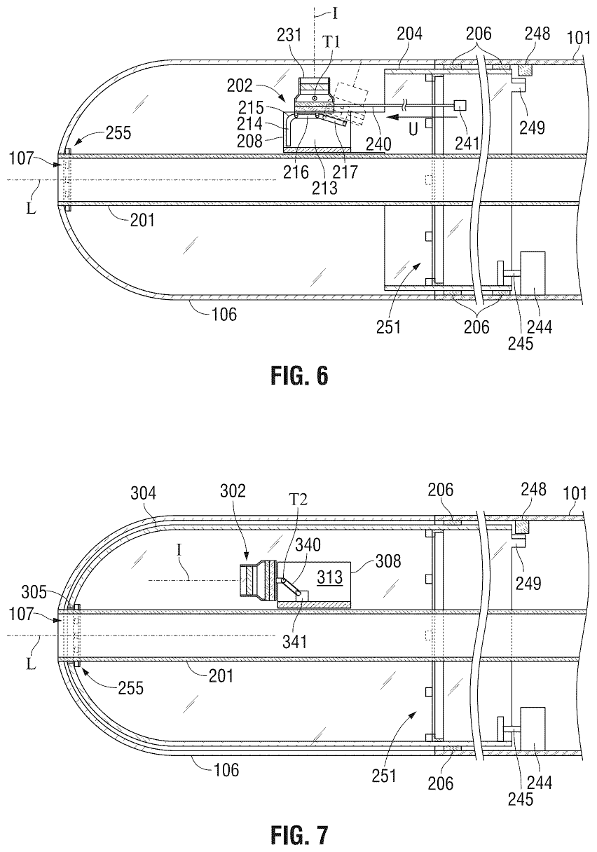

The imaging apparatus shown in FIGS. 2-6 includes an image sensor assembly 202 mounted on a structure made up of a first articulating structure and a second articulating structure. In this illustrated embodiment, the first articulating structure includes an axial articulation tube 204 mounted on shaft 101 on one or more bearings 206 (visible in FIGS. 3 and 6) with a longitudinal axis of the axial articulation tube 204 aligning with the longitudinal axis L of shaft distal end portion 106. The bearings 206 may comprise any suitable devices such as bushings or bearings with roller elements, for example, that allow the axial articulation tube 204 to rotate about the longitudinal axis L.

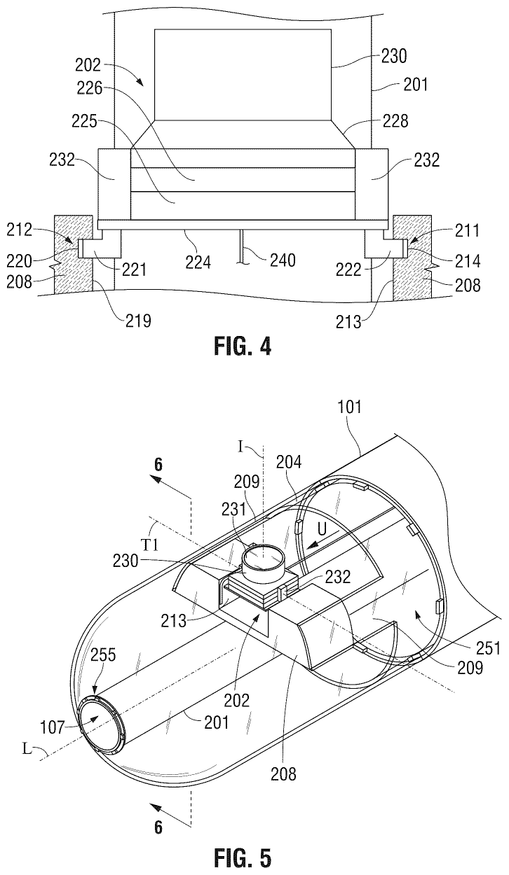

The second articulating structure is mounted on axial articulation tube 204 and, in the example apparatus shown in FIGS. 2-6, comprises a pivot base 208. Pivot base 208 is mounted on two circumferentially spaced apart extensions 209 that extend from axial articulation tube 204 and form part of the first articulating structure. In this embodiment axial articulation tube 204 and extensions 209 may or may not be formed from a transparent material. Pivot base 208 includes a first base pivot structure 211 that is visible in the views of FIGS. 2-6 and a second base pivot structure 212 that is only visible in part in the view of FIG. 4. As shown best in FIGS. 3 and 6, first base pivot structure 211 is made up of a track including a series of connected segments formed in a surface 213 of pivot base 208. The track segments include a transverse portion 214, and arcuate connecting portion 215, and a longitudinal portion 216 with an inclined part 217. Second pivot structure 212 is made up of a corresponding track including a series of connected segments formed in surface 219 of pivot base 208, although only the transverse portion 220 is visible in the figures in FIG. 4.

Image sensor assembly 202 in this embodiment includes a first sensor pivot structure adapted to cooperate with the first base pivot structure and a second sensor pivot structure adapted to cooperate with the second base pivot structure to allow the image sensor assembly to rotate about a lateral articulation axis T1. This rotation allows an imaging axis I of the image sensor assembly 202 to rotate about lateral articulation axis T1 as will be described further below particularly in connection with FIGS. 3 and 6. As shown perhaps best in FIGS. 2 and 5, lateral articulation axis T1 extends transverse to the longitudinal axis L, perpendicular in this example (and thus represented as a point in the views of FIGS. 3 and 6. In the embodiment shown in FIGS. 2-6 each of the first and second sensor pivot structure includes fore and aft projections, 221 and 222, respectively, on each lateral side of image sensor assembly 202 (as indicated particularly in FIGS. 3, 4, and 6). These projections 221 and 222 each cooperate with the respective track arrangement on pivot base 208 described above to facilitate articulation about the lateral articulation axis T1 shown in FIGS. 2, 3, 5 and 6.

Image sensor assembly 202 shown in FIGS. 2-6 includes an imaging arrangement including a printed circuit board (“PCB”) 224 on which is mounted an imaging device 225 that in this case may comprise, for example, a charge coupled device (“CCD”) having a transparent cover 226. Image sensor assembly 202 also includes an adapter 228 which connects the rectangular image sensing device 225 and cover 226 to an objective lens assembly having a cylindrical sleeve 230. The objective lens assembly includes one or more lenses 231 mounted in sleeve 230 and allows image sensor assembly 202 to obtain an image within a field of view about the imaging axis I. The illustrated image sensor assembly also includes a sensor assembly light source in the form of two LED lamps 232 mounted on PCB 224.

It will be appreciated by those familiar with imaging devices (such as device 225 in FIGS. 2-6) that these devices may be accompanied by electronic components such as transistors, capacitors, resistors, and regulators for example. Additionally, imaging device 225 and its accompanying electronic components require electrical power and means for communicating image data to be processed for producing the collected images. The required operating power and data transmission may be provided through a suitable electrical cable. These accompanying electronic components and the power/data cable are omitted from the present drawings in order to more clearly illustrate the various features of the claimed imaging apparatus. Those skilled in the art will appreciate that the electronic components and power/data cable may be connected to or included with image sensor assembly 202 in any number of fashions. For example, some embodiments of an articulating imaging apparatus may include the electronic components mounted on the side of PCB 224 opposite the side on which imaging device 225 is mounted. The power/data cable may also be connected to the back side of PCB 224 to provide operating power to image sensor assembly 202 and allow image data to be communicated from the image sensor assembly to processing equipment remote from the shaft distal end portion 106. However, the disclosed technology is not limited to any particular mounting arrangement for electronic components and a power/data cable that may accompany imaging device 225. Any accompanying electronic components and the power/data cable need only be mounted to provide the required function and allow the movement of image sensor assembly 202 across its desired range of movement.

FIGS. 2, 3, 5, and 6 also show that image sensor assembly 202 is connected to a push wire 240 extending longitudinally along instrument shaft 101. Push wire 240 comprises part of an articulation control assembly in this embodiment and is used together with linear actuator 241 shown in FIGS. 3 and 6 to control the articulation of image sensor assembly 202 about lateral articulation axis T1 as will be described below. FIGS. 3 and 6 also show a motor 244 and drive linkage 245 that are also part of the articulation control assembly in this embodiment and are used to control the articulation of image sensor assembly 202 about longitudinal axis L as will be described below. A stop 248 is included on the inside surface of shaft 101 in this embodiment and cooperates with a tab 249 extending from axial articulation tube 204 to prevent axial articulation tube 204 from being rotated more than 360° about longitudinal axis L.

An additional illumination element may be included with an imaging apparatus in accordance with the disclosed technology. The embodiment shown in FIGS. 2-6, for example, shows a base light source 251 made up of a number of individual LEDs 252 spaced apart circumferentially about a mounting ring 253 connected to the inner surface of axial articulation tube 204. LEDs 252 are thus positioned in this embodiment to emit light that may pass through the transparent material of distal end portion 106 to illuminate a wide area around the distal end portion. Alternatively to the position of the base light source 251 in FIGS. 2-6, a similar arrangement of LEDs may be mounted directly to the inner surface of shaft 101 or the inner surface of distal end portion 106 to provide a similar wide area illumination around the distal end portion.

The instrument shown in FIGS. 2-6 also includes a distal tip light source shown generally at 255 adjacent to working channel opening 109 at the far distal end of distal end portion 106. This example distal tip light source includes a number of individual LEDs 256 space apart circumferentially about a mounting ring 257 that may be connected on the outer surface of working channel tube 201.

Base light source 251 and/or distal tip light source 255 may be used in addition to LEDs 232 included on image sensor assembly 202 in this embodiment. Alternative embodiments may omit LEDs 232 and rely on base light source 251 and/or distal tip light source 255 for illumination needed for imaging or for procedures conducted through working channel 201. The light emitted by the various LEDs 232, 252, and 256 may be in any desired spectrum. Also, some of these LEDs may emit in a first spectrum while others may emit in a second, different spectrum. It should also be appreciated that while the light emitting elements are described here as LEDs, the disclosed technology is not limited to any particular light emitting technology for the light emitting elements. Regardless of the light emitting technology employed by the light emitting elements (corresponding to LEDs 232, 252, and 256) electrical operating power that may be required by the light emitting elements may be provided from a power source through any suitable arrangement of conduction paths (not shown) through shaft 101. For example, a suitable electrical conductor arrangement for distal tip light source 255 may be supported on working channel tube 201, particularly on the exterior surface of that tube. Because the illustrated base light source 251 rotates with respect to shaft 101, a suitable slip ring arrangement (not shown) may be used to provide operating power to the LEDs 252 of base light source 251.

FIGS. 2-6 may now be used to describe the articulation of image sensor assembly 202 within instrument distal end portion 106. In the position of image sensor assembly 202 shown in FIGS. 2-4, the image sensor assembly is oriented about lateral articulation axis T1 so that imaging axis I is aligned essentially parallel to longitudinal axis L of shaft distal end portion 106. Imaging axis I in this position is offset from longitudinal axis L due to the positioning of image sensor assembly 202 in the annular space between working channel 201 and distal end portion 106. The disclosed technology allows image sensor assembly 202 to be reoriented within shaft distal end portion 106 to change the field of view without moving the shaft distal end portion 106. In particular, from the position shown in FIGS. 2-4, image sensor assembly 202 may be moved to the position shown in FIGS. 5 and 6 in which imaging axis I lies at an angle of approximately 90° to longitudinal axis L. It is the ability of the projections 221 and 222 to move along the tracks comprising the first and second base pivot structures 211 and 212 that facilitates this lateral axis articulation in this embodiment. Push wire 240 may be manipulated longitudinally to place image sensor assembly 202 in the desired orientation about lateral articulation axis T1 within the instrument shaft distal end portion 106. As compared to the position shown best in FIGS. 2 and 3, push wire 240 may be pulled in the direction D in FIGS. 2 and 3 to move image sensor assembly 202 from the position shown in those figures to the position shown in FIGS. 5 and 6. Image sensor assembly 202 may be returned to the position shown in FIGS. 2-4 by moving push wire 240 in the opposite direction U shown in FIGS. 5 and 6. Of course image sensor assembly 202 may be stopped at any position between the position of FIG. 2 and the position of FIG. 5 in order to obtain a view at that particular location. Additionally, the inclined segments 217 of each track/base pivot structure 211 and 212 allow image sensor assembly 202 to be positioned as shown in phantom lines in FIG. 6 in which imaging axis I extends somewhat back away from distal end portion 106 (for example, at an angle of 120° from longitudinal axis L.

Since axial articulation tube 204 is mounted for rotation about longitudinal axis L, image sensor assembly 202 (and its imaging axis I) may be rotated to any position 360° around longitudinal axis L without changing the position of the instrument shaft 101 or distal end portion 106. Thus the instrument operator is able to view a large area all without changing the position of the instrument relative to that area. For example, from the position shown in FIG. 6 with the imaging axis I pointing upwardly in the orientation of the drawing, axial articulation tube 204 may be rotated 180° about longitudinal axis L using motor 244 and drive linkage 245 so that imaging sensor assembly 202 resides below working channel 201 in the orientation of the drawing and imaging axis I points downwardly. It is further noted that the position of image sensor assembly 202 to the side of working channel 201 results in image sensor assembly 202 rotating around working channel 201 as axial articulation tube 204 is rotated about longitudinal axis L. Regardless of the angular orientation of axial articulation tube 204 about longitudinal axis L, the first and second base pivot structures 211 and 212 cooperate with the first and second sensor pivot structures each made up of projections 221 and 222 so that throughout the rotation of image sensor assembly 202 and imaging axis I about the lateral articulation axis TI, image sensor assembly 202 remains within the confines of the sensor area defined between the external surface of working channel 201 and the internal surface of shaft distal end portion 106.

As shown in FIGS. 3 and 6 of the illustrated representative embodiment, linear actuator 241 controls push wire 240 and thus the position of image sensor assembly 202 along the tracks representing the first and second base pivot structures 211 and 212. Linear actuator 241 may comprise any suitable device for imparting the desired motion to push wire 240 and may be located at any point that does not interfere with the rotation of axial articulation tube 204 about longitudinal axis L. FIGS. 3 and 6 also show motor 244 and drive linkage 245 that operate together to control the rotation of axial articulation tube 204 about longitudinal axis L. Motor 244 may be any suitable device (such as a stepper motor) for rotating drive linkage 245 about its rotational axis R. Drive linkage 245 may rely on a frictional engagement with the inner surface of axial articulation tube 204 or may cooperate with an internal gear (not shown) extending circumferentially around the inner surface of inner tube. Linear actuator 241 and motor 244 may be operated through controls located on handle 102 shown in FIG. 1. For example, one of the buttons shown at controls 110 in FIG. 1 may comprise a toggle button that may be depressed to one side to drive linear actuator 241 (and thus image sensor assembly 202) in one direction and depressed to the opposite side to drive the linear actuator in the opposite direction. Similarly, another one of the buttons shown at controls 110 in FIG. 1 may comprise a toggle button that may be depressed to one side to drive motor 244 (and thus axial articulation tube 204) in one rotational direction and depressed to the opposite side to drive the motor in the opposite rotational direction. Of course, in the case of either linear actuator 241 or motor 244, the device will typically be associated with suitable control circuitry (not shown) that receives signals from the operator activated button or other control device as an input and directs suitable driving signals to the respective device, 241 and 244.

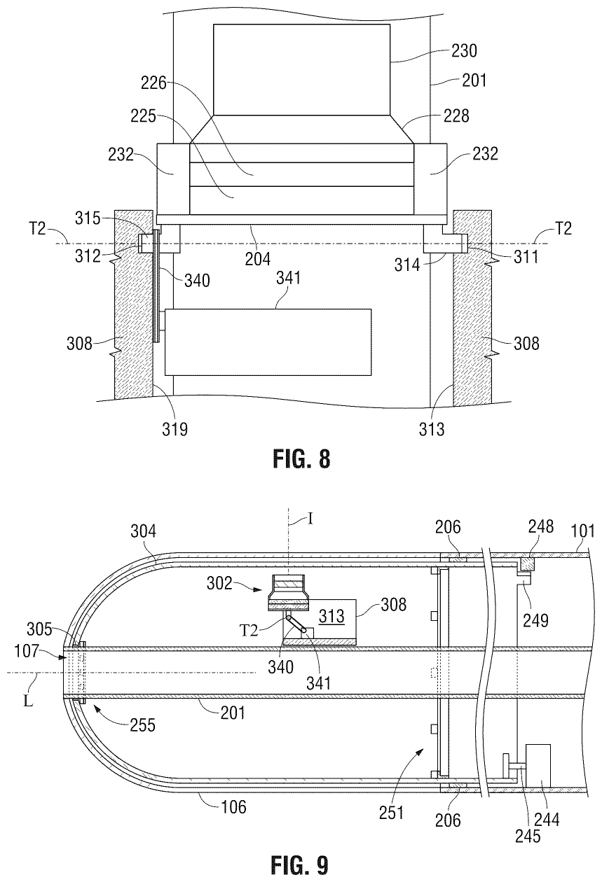

FIGS. 7-9 show an alternative embodiment of an imaging apparatus with a different first and second articulating structures as compared to those shown in FIGS. 2-6, and a somewhat different image sensor assembly 302. The embodiment shown in FIGS. 7-9 includes the same instrument shaft 101, shaft distal end portion 106, working channel opening 107, and working channel tube 201 shown in the embodiment of FIGS. 2-6, as well as the same imaging device 225 and related components for collecting an image along imaging axis I. However, the first articulating structure in FIGS. 7-9 comprises an axial articulation tube 304 that is made of a transparent material extending distally past image sensor assembly 302 with a curved end part that terminates at an opening defined by edge 305 through which working channel tube 201 passes to reach the working channel opening 107. The second articulating structure in the embodiment of FIGS. 7-9 comprises a pivot base 308 that is mounted directly on the axial articulation tube 304 (rather than being mounted on extensions 209 as shown in FIG. 2) and allows the image sensor assembly 302 to be rotated about a lateral articulation axis T2. Pivot base 308 includes laterally spaced apart pivot receivers 311 and 312 formed on base surfaces 313 and 319, respectively as shown in FIG. 8. These pivot receivers 311 and 312 represent first and second base pivot structures in this embodiment. Image sensor assembly 302 includes a pivot element 314 rotatably received in pivot receiver 311 and a pivot element 315 rotatably received in pivot receiver 312. This cooperation between pivot receivers 311 and 312 and pivot elements 314 and 315 allows image sensor assembly 302 to be pivoted about lateral articulation axis T2 from the orientation shown in FIGS. 7 and 8 to the orientation shown in FIG. 9 or any point in between. The pivot structure shown in FIGS. 7-9 also allows image sensor assembly 302 to be pivoted further than 90° to longitudinal axis L so that imaging axis I is directed somewhat away from the distal end of the instrument and back toward the proximal end of shaft 101.

The orientation of image sensor assembly 302 in the example of FIGS. 7-9 is controlled through an articulation control arrangement comprising a motor 341 and drive linkage 340 that comprises a sprocket or pulley system operating between a shaft of motor 341 and pivot element 314. Motor 341 may comprise any suitable device (such as a stepper motor) for imparting the desired rotation to image sensor assembly 302 and holding the assembly in a desired rotational position about lateral articulation axis T2. Motor 341 may be controlled through a suitable motor control circuit (not shown) that takes an input from a control device on the instrument handle 102 shown in FIG. 1. Similarly to the previously described embodiment, a button included with controls 110 in FIG. 1 may comprise a toggle button that may be depressed to one side to drive motor 341 in one direction and depressed to the opposite side to drive the motor in the opposite rotational direction. As shown in FIGS. 7 and 9, rotation of axial articulation tube 304 (and thus rotation of image sensor assembly 302 about longitudinal axis L) may be controlled via the same arrangement of motor 244 and drive linkage 245 shown in the embodiments of FIGS. 3 and 6.

Although the entire axial articulation tube 304 is illustrated as being transparent, it need not be transparent around its entire circumference as shown in the present figures. Rather, axial articulation tube 304 may be transparent only at the end and along the side to which image sensor assembly 302 may be turned about lateral articulating axis T2, so as not to block the field of view about imaging axis I regardless of the orientation of the image sensor assembly 302 about the lateral articulation axis T2.

In implementations employing an elongated tube (such as axial articulation tubes 204 and 304) for the first articulating structure, the tube may include a proximal end adapted to extend to a handle of the instrument when the imaging apparatus is mounted in the operating position in the instrument shaft. Thus the motor 244 may be mounted in handle 102 in FIG. 1. This is in contrast to the position of the motor 244 shown in the present figures within the instrument shaft 101 at a location near the distal end portion 106. It is also possible for the motor 244 to be mounted in an instrument handle with a torque transmission element extending from the motor to the first articulation structure. Some embodiments of may include a first articulation structure that may be manually turned through a suitable linkage mounted on the instrument handle, and thus omit a motor such as motor 244. Similarly, push wire 240 in the embodiment of FIGS. 2-6 may be manipulated with a suitable manually operated control on the handle 102 rather than through linear actuator 241.

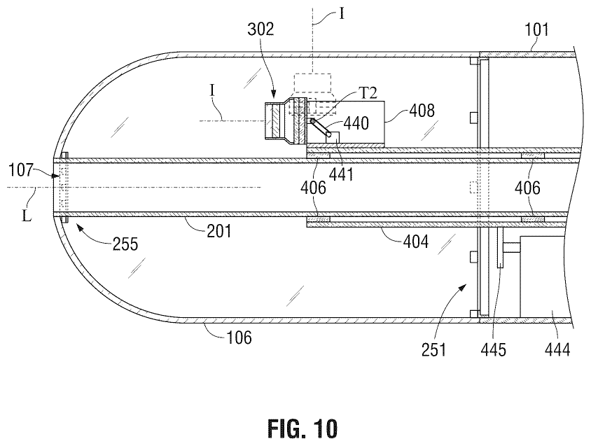

FIG. 10 shows the distal end of an instrument including an imaging apparatus in accordance with the disclosed technology having a different first articulation structure as compared to the first articulation structure comprising axial articulation tube 204 in the embodiment of FIGS. 2-6 and 304 in the embodiment of FIGS. 7-9. The instrument shown in FIG. 10 includes the instrument shaft 101, transparent distal end portion 106, working channel opening 107, and working channel 201 as in the previously described embodiments. The imaging apparatus shown in the embodiment of FIG. 10 includes the same image sensor assembly 302 shown in the embodiment of FIGS. 7-9. However, the embodiment of FIG. 10 includes a first articulation structure comprising an axial articulation tube 404 that is supported on working channel 201. In particular, axial articulation tube 404 is supported on working channel 201 through bearings 406 that allow the axial articulation tube 404 to rotate with respect to working channel 201 about longitudinal axis L. This is in contrast to the two previously described embodiments in which the respective axial articulation tube 204 and 304 is mounted for rotation on the instrument shaft 101. In view of this mounting of axial articulation tube 404 on working channel 201, the embodiment of FIG. 10 includes a pivot base 408 mounted on the outer surface of axial articulation tube 404.

The articulation of image sensor assembly 302 and imaging axis I about lateral articulation axis T2 in the embodiment of FIG. 10 (axis T2 extending perpendicular to the plane of FIG. 10 and thus represented by a point in the figure) is identical to the articulation described above in connection with FIGS. 7-9. Namely, image sensor assembly 302 includes the pivot elements 314 and 315 as shown in FIG. 8 that cooperate with pivot receivers similar to receivers 311 and 312 in FIG. 8 to allow the image sensor assembly 302 to pivot at least from the position shown in solid lines in FIG. 10 to the position shown in phantom lines in that figure. The rotation about lateral articulation axis T2 is driven in the example of FIG. 10 with motor 441 through linkage 440 which correspond respectively to motor 341 and linkage 340 in the embodiments of FIGS. 7-9.

In view of the position of axial articulation tube 404 on working channel 201, the embodiment of FIG. 10 has a somewhat different arrangement for driving the rotation about longitudinal axis L. FIG. 10 shows a motor 444 with a linkage 445 such as a gear or other device adapted to cooperate with a corresponding gear or device on the exterior of axial articulation tube 404. A suitable stop arrangement (not shown in FIG. 10) may be provided to prevent axial articulation tube from being rotated more than 360° to avoid damaging electrical connections to image sensor assembly 302 and lighting elements in the apparatus.

In the embodiment of FIG. 10, axial articulation tube 404 may alternatively be rotated manually or using a motor remote from the distal end portion 106 as described above in connect with axial articulation tubes 204 and 304. As for the articulation of image sensor assembly 302 about lateral articulation axis T2, this may be controlled through arrangements other than motor 341 and drive linkage 340 shown in the illustrated embodiments, even manually via suitable pivot control wires or elements (not shown) extending through shaft 101 to a handle such as handle 102 shown in FIG. 1

The various components of an articulating image sensing arrangement may be formed from any suitable material or combination of materials. The materials should be selected for compatibility with the instrument with which the apparatus is to be used, and the environments to which the instrument may be subjected. For example, for use in endoscopes, the components of the articulating image sensing arrangement should be compatible with materials and conditions used in sterilizing procedures for such endoscopes. Where different components are connected such as the connection between pivot base 208 and extensions 209 or between pivot base 308 and axial articulation tube 304, the connection may be made in any suitable manner such as with a suitable adhesive. The pivot base 408 may also be attached to axial articulation tube 404 with a suitable adhesive or the two components may be made in one piece using a suitable additive manufacturing process. Other parts of the disclosed technology may be produced by additive manufacturing as well. Also, although a CCD-type imaging device is referenced in the representative examples described above, any suitable imaging device may be employed within the scope of the disclosed technology. For example, imaging device 225 may comprise a CMOS imaging device or any other type of imaging device.

Numerous variations are possible in an imaging apparatus and instrument in accordance with the disclosed technology. For example, although the illustrations show a certain position of the respective image sensor assembly long longitudinal axis L, the image sensor assembly could be at any suitable axial position within distal end portion 106. The various exemplary features as set out in the following claims also encompass numerous variations in the sensor assemblies 202 and 302. Image sensor assembly 202 is similar to that shown at 202 in FIGS. 2-6 of U.S. Pat. No. 10,517,470 which is hereby incorporated herein by reference. Any of the compatible variations described in U.S. Pat. No. 10,517,470 regarding image sensor assembly 202 of FIGS. 2-6 in that reference apply equally to image sensor assembly 202 in the present drawings. Image sensor assembly 302 in the present drawings is similar to that shown at 202 in FIGS. 7-9 of U.S. Pat. No. 10,517,470. Any of the compatible variations described in U.S. Pat. No. 10,517,470 regarding image sensor assembly 202 of FIGS. 7-9 in that reference apply equally to image sensor assembly 302 in the present drawings.

As used herein, whether in the above description or the following claims, the terms “comprising,” “including,” “carrying,” “having,” “containing,” “involving,” and the like are to be understood to be open-ended, that is, to mean including but not limited to. Also, it should be understood that the terms “about,” “substantially,” and like terms used herein when referring to a dimension or characteristic of a component indicate that the described dimension/characteristic is not a strict boundary or parameter and does not exclude variations therefrom that are functionally similar. At a minimum, such references that include a numerical parameter would include variations that, using mathematical and industrial principles accepted in the art (e.g., rounding, measurement or other systematic errors, manufacturing tolerances, etc.), would not vary the least significant digit.

Any use of ordinal terms such as “first,” “second,” “third,” etc., in the following claims to modify a claim element does not by itself connote any priority, precedence, or order of one claim element over another, or the temporal order in which acts of a method are performed. Rather, unless specifically stated otherwise, such ordinal terms are used merely as labels to distinguish one claim element having a certain name from another element having a same name (but for use of the ordinal term).

The term “each” may be used in the following claims for convenience in describing characteristics or features of multiple elements, and any such use of the term “each” is in the inclusive sense unless specifically stated otherwise. For example, if a claim defines two or more elements as “each” having a characteristic or feature, the use of the term “each” is not intended to exclude from the claim scope a situation having a third one of the elements which does not have the defined characteristic or feature.

The above-described exemplary embodiments are intended to illustrate the principles of the disclosed technology, but not to limit the scope of the disclosed technology. Various other embodiments and modifications to these exemplary, non-exhaustive embodiments may be made by those skilled in the art without departing from the scope of the disclosed technology. For example, in some instances, one or more features disclosed in connection with one embodiment can be used alone or in combination with one or more features of one or more other embodiments. More generally, the various features described herein may be used in any working combination.

### LIST OF REFERENCE SIGNS

100 instrument
101 shaft
102 handle
104 proximal end
105 distal end
106 distal end portion
107 working channel opening
108 cable
109 port
110 control
201 working channel
202 image sensor assembly
204 axial articulation tube
206 bearing
208 pivot base
209 extension
211 first base pivot structure
212 second base pivot structure
213 surface
214 transverse portion
215 arcuate connecting portion
216 longitudinal portion
217 inclined part
219 surface
220 transverse portion
221 projection
222 projection
224 printed circuit board (“PCB”)
225 imaging device
226 transparent cover
228 adapter
230 cylindrical sleeve
232 LED
240 push wire
241 linear actuator
244 motor
245 drive linkage
248 stop
249 tab
251 base light source
252 LED
253 mounting ring
255 distal tip light source
256 LED
257 mounting ring
302 image sensor assembly
304 axial articulation tube
305 edge
308 pivot base
311 pivot receiver
312 pivot receiver
313 base surface
314 pivot element
315 pivot element
319 base surface
340 drive linkage
341 motor
404 axial articulation structure
406 bearing
408 pivot base
440 linkage
441 motor
444 motor
445 linkage
L longitudinal axis
T1 lateral articulation axis
I imaging axis
D direction
U opposite direction (to D)
R rotational axis
T2 lateral articulation axis

1. An instrument including:
an instrument shaft having a distal end portion that is transparent, the distal end portion also having a working channel opening;
a working channel tube extending through the instrument shaft to the working channel opening with a sensor area being defined between an external surface of the working channel tube and an internal surface of the distal end portion;
a first articulating structure positioned within the instrument shaft for rotation about a longitudinal axis (L) of the distal end portion of the instrument shaft;
a second articulating structure mounted on the first articulating structure;
an image sensor assembly having an imaging axis (I), the image sensor assembly being mounted on the second articulating structure to facilitate rotation of the imaging axis (I) about a lateral articulation axis (T1, T2) extending transverse to the longitudinal axis (L) of the distal end portion of the instrument shaft while maintaining the image sensor assembly within the sensor area; and
an articulation control assembly configured to control rotation of the first articulating structure about the longitudinal axis (L) of the distal end portion of the instrument shaft and to control the articulation of the image sensor assembly about the lateral articulation axis (T1, T2).

2. The instrument of claim 1 wherein the first articulating structure includes an axial articulation tube within the instrument shaft with a longitudinal axis of the axial articulation tube aligning with the longitudinal axis (L) of the distal end portion of the instrument shaft, the axial articulation tube being mounted within the instrument shaft for rotation about the longitudinal axis (L) of the distal end portion of the instrument shaft.

3. The instrument of claim 2 wherein the axial articulation tube is connected to the working channel tube through an axial articulation tube bearing structure so as to be supported by the working channel tube.

4. The instrument of claim 3 wherein:
the second articulation structure comprises a pivot base mounted on the axial articulation tube, the pivot base including a first base pivot structure and a second base pivot structure;
the image sensor assembly includes a first sensor pivot structure and a second sensor pivot structure; and
the first base pivot structure cooperates with the first sensor pivot structure and the second base pivot structure cooperates with the second sensor pivot structure to facilitate the rotation of the imaging axis (I) about the lateral articulation axis (T1, T2).

5. The instrument of claim 2 wherein the axial articulation tube is connected to the instrument shaft through an axial articulation tube bearing structure so as to be supported by the instrument shaft.

6. The instrument of claim 5 wherein:
the second articulation structure comprises a pivot base mounted on the axial articulation tube, the pivot base including a first base pivot structure and a second base pivot structure;
the image sensor assembly includes a first sensor pivot structure and a second sensor pivot structure; and
the first base pivot structure cooperates with the first sensor pivot structure and the second base pivot structure cooperates with the second sensor pivot structure to facilitate the rotation of the imaging axis (I) about the lateral articulation axis (T1, T2).

7. The instrument of claim 6 wherein the pivot base is mounted on the axial articulation tube on two circumferentially spaced apart extensions that extend from a proximal portion of the axial articulation tube in an area between a distal tip of the instrument shaft and the axial articulation tube bearing structure.

8. The instrument of claim 1 further including a distal tip light source located within the distal end portion and external to the working channel tube at a location along the longitudinal axis (L) of the distal end portion of the instrument shaft between a distal tip of the instrument shaft and the lateral articulation axis (T1, T2).

9. The instrument of claim 1 further including a base light source located within the distal end portion at a location at a first distance from a distal tip of the instrument shaft, the first distance being greater than a distance between the distal tip of the instrument shaft and the lateral articulation axis (T1, T2).

10. The instrument of claim 1 further including:
a sensor assembly light source mounted on the image sensor assembly so as to articulate with the image sensor assembly about the lateral articulation axis (T1, T2), and
at least one of,
(i) a distal tip light source located within the distal end portion and external to the working channel tube at a location along the longitudinal axis (L) of the distal end portion of the instrument shaft between a distal tip of the instrument shaft and the lateral articulation axis (T1, T2), and
(ii) a base light source located within the distal end portion at a location at a first distance from the distal tip of the instrument shaft, the first distance being greater than a distance between the distal tip of the instrument shaft and the lateral articulation axis (T1, T2).

11. An imaging apparatus for an instrument having an elongated instrument shaft and a working channel tube, the elongated instrument shaft having a distal end portion that is transparent and includes a working channel opening, the working channel tube extending through the instrument shaft to the working channel opening, the imaging apparatus including:
a first articulating structure adapted to be positioned within the instrument shaft in an operating position for rotation about a longitudinal axis (L) of the distal end portion of the instrument shaft;
a second articulating structure mounted on the first articulating structure; and
an image sensor assembly having an imaging axis (I), the image sensor assembly being mounted on the second articulating structure to facilitate rotation of the imaging axis (I) about a lateral articulation axis (T1, T2) extending transverse to the longitudinal axis (L) of the distal end portion of the instrument shaft while maintaining the image sensor assembly within a sensor area defined between an external surface of the working channel tube and in internal surface of the distal end portion.

12. The imaging apparatus of claim 11 wherein the first articulating structure includes an axial articulation tube adapted to be positioned within the instrument shaft with a longitudinal axis of the axial articulation tube aligning with the longitudinal axis (L) of the distal end portion of the instrument shaft, the axial articulation tube adapted to be mounted within the instrument shaft for rotation about the longitudinal axis (L) of the distal end portion of the instrument shaft.

13. The imaging apparatus of claim 12 further including an axial articulation tube bearing structure adapted to connect the axial articulation tube to the working channel tube so as to support the axial articulation tube on the working channel tube.

14. The imaging apparatus of claim 13 wherein:
the second articulation structure comprises a pivot base mounted on the axial articulation tube, the pivot base including a first base pivot structure and a second base pivot structure;
the image sensor assembly includes a first sensor pivot structure and a second sensor pivot structure; and
the first base pivot structure cooperates with the first sensor pivot structure and the second base pivot structure cooperates with the second sensor pivot structure to facilitate the rotation of the imaging axis (I) about the lateral articulation axis (T1, T2).

15. The imaging apparatus of claim 12 further including an axial articulation tube bearing structure adapted to connect the axial articulation tube to the instrument shaft so as to support the axial articulation tube on the instrument shaft.

16. The imaging apparatus of claim 15 wherein:
the second articulation structure comprises a pivot base mounted on the axial articulation tube, the pivot base including a first base pivot structure and a second base pivot structure; and
the image sensor assembly includes a first sensor pivot structure and a second sensor pivot structure; and
the first base pivot structure cooperates with the first sensor pivot structure and the second base pivot structure cooperates with the second sensor pivot structure to facilitate the rotation of the imaging axis (I) about the lateral articulation axis (T1, T2).

17. The imaging apparatus of claim 16 wherein the pivot base is mounted on the axial articulation tube on two circumferentially spaced apart extensions that extend from a proximal portion of the axial articulation tube.

18. The imaging apparatus of claim 11 further including a distal tip light source mounted on the first articulating structure at a distal end of the first articulating structure.

19. The imaging apparatus of claim 11 further including a base light source mounted within the first articulating structure between a proximal end of the first articulating structure and lateral articulation axis (T1, T2).

20. The imaging apparatus of claim 11 further including:
a sensor assembly light source mounted on the image sensor assembly so as to articulate with the image sensor assembly about the lateral articulation axis (T1, T2), and at least one of,
(i) a distal tip light source mounted on the first articulating structure at a distal end of the first articulating structure, and
(ii) a base light source mounted within the first articulating structure between a proximal end of the first articulating structure and lateral articulation axis (T1, T2).
